(19) United States
(12) Patent Application Publication
(10) Pub. No.: US 2011/0151469 A1
(43) Pub. Date: Jun. 23, 2011

(54) INTERFERON EPSILON (IFNE1) AS A MARKER FOR TARGETED CANCER THERAPY

(75) Inventors: SUNITA SHANKAR, ANN ARBOR, MI (US); URVI VED, FREMONT, CA (US); SOMESH SHARMA, MUMBAI (IN)

(73) Assignee: PIRAMAL LIFE SCIENCES LIMITED, MUMBAI (IN)

(21) Appl. No.: 12/964,417

(22) Filed: Dec. 9, 2010

Related U.S. Application Data
(63) Continuation-in-part of application No. PCT/IB2008/052278, filed on Jun. 10, 2008.

(51) Int. Cl. C12Q 1/68; G01N 33/574

(52) U.S. Cl. 435/6.13; 435/7.8

(57) ABSTRACT

The present invention relates to a method employing Interferon Epsilon (IFNE1) as a therapeutic response, prognostic, or pharmacodynamic marker for cancer chemotherapeutic treatment involving the use of cyclin dependent kinase (CDK) inhibitors. The inventors have identified IFNE1 as a biomarker transcript that is upregulated when cancer cells are treated with CDK inhibitors. In an embodiment, the method of the invention includes measuring a level of IFNE1 mRNA or IFNE1 protein in a subject's tumor, blood or other tissue. An increase in the level of IFNE1 compared to control level can indicate that the CDK inhibitor has produced a therapeutic response or can determine whether a tumor is sensitive to a CDK inhibitor.

### FIELD OF THE INVENTION

The present invention relates to a method employing Interferon Epsilon (IFNE1) as a therapeutic response, prognostic, or pharmacodynamic marker for cancer chemotherapeutic treatment involving the use of cyclin dependent kinase (CDK) inhibitors. The inventors have identified IFNE1 as a biomarker transcript that is upregulated when cancer cells are treated with CDK inhibitors. In an embodiment, the method of the invention includes measuring a level of IFNE1 mRNA or IFNE1 protein in a subject's tumor, blood or other tissue. An increase in the level of IFNE1 compared to control level can indicate that the CDK inhibitor has produced a therapeutic response or can determine whether a tumor is sensitive to a CDK inhibitor.

### BACKGROUND OF THE INVENTION

Interferons (IFNs) are a class of natural proteins produced by the cells of the immune systems of most animals in response to challenges by foreign agents such as viruses, bacteria, parasites. They belong to the large class of glycoproteins known as cytokines. There are three classes of IFNs: IFN-α secreted by leukocytes, IFN-β secreted by fibroblasts and IFN-γ secreted by T-cells and natural killer lymphocytes. IFNs-α, β and ω are known to induce MHC Class I antigens, and are referred to as type I IFNs, while IFN-γ induces MHC Class II antigen expression, and is also referred to as type II IFN.

Nucleic acids encoding novel human Interferon Epsilon (IFNE1 or IFNε), its variants and derivatives, and methods for their recombinant production are described in U.S. Pat. Nos. 6,569,420; 6,200,780; 6,300,475; and 6,299,869, incorporated herein by reference. These patents also describe the use of IFNE1 in the inhibition of neoplastic cell growth, treatment of viral infections and in general upregulation of the immune system and thus the therapeutic potential of IFNE1 protein in the treatment of related conditions and disorders.

Antibodies specifically binding various interferons are known in the art. An antibody that specifically binds to an IFNE1 polypeptide and method of determining the same are claimed and described in U.S. Pat. No. 6,299,877, incorporated herein by reference.

The present invention describes the use of IFNE1 as a therapeutic response and prognostic marker for chemotherapeutic agents that target cyclin dependent kinases.

### SUMMARY OF THE INVENTION

The present invention relates to a method of evaluating an agent used to treat cancer. This embodiment of the method can include comparing expression of a biomarker transcript before administering the therapeutic agent to the expression of the biomarker transcript after administering the agent. In an aspect, increased expression of the biomarker transcript indicates that the therapeutic agent is effective in treating the cancer or that a cancer cell will respond to treatment with the agent.

In an embodiment, the present invention relates to a method of evaluating administration of an agent used to treat cancer. This embodiment of the method can include measuring an amount of a biomarker transcript in a cancer cell, contacting the cancer cell with the agent, and measuring amount of the biomarker transcript in the cancer cell after administering the agent. Comparing the amount of the biomarker transcript measured after administering the therapeutic agent to the amount of biomarker transcript measured before administering the therapeutic agent provides the evaluation. An increase in the amount of biomarker transcript after administration of the therapeutic agent indicates that the therapeutic agent is effective in treating cancer. In an embodiment, the therapeutic agent is a cyclin-dependent kinase (CDK) inhibitor and the biomarker transcript is IFNE1 (e.g., mRNA or protein).

In an embodiment, the present invention relates to a method of evaluating whether a cyclin-dependent kinase (CDK) inhibitor will inhibit growth of a cancer cell. This embodiment of the method includes measuring an amount of a biomarker transcript in the cancer cell before administering the CDK inhibitor, contacting the cancer cell with the CDK inhibitor, and measuring amount of the biomarker transcript in the cancer cell after administering the CDK inhibitor. Comparing the amount of the biomarker transcript measured after administering the CDK inhibitor to the amount of the biomarker transcript measured before administering the CDK inhibitor provides the evaluation. An increase in amount of the biomarker transcript indicates that the CDK inhibitor will inhibit growth of a cancer cell. In an embodiment, the biomarker transcript is IFNE1 (e.g., mRNA or protein).

In an embodiment, the present invention relates to a method of evaluating whether a cyclin-dependent kinase (CDK) inhibitor produces a therapeutic response in treating cancer. This embodiment of the method includes measuring an amount of a biomarker transcript in the cancer cell before administering the CDK inhibitor, contacting the cancer cell with the CDK inhibitor, and measuring amount of the biomarker transcript in the cancer cell after administering the CDK inhibitor. Comparing the amount of the biomarker transcript measured after administering the CDK inhibitor to the amount of the biomarker transcript measured before administering the CDK inhibitor provides the evaluation. An increase in the expression of the biomarker transcript after administration of the CDK inhibitor indicates that the CDK inhibitor produces a therapeutic response in treating the cancer. In an embodiment, the biomarker transcript is IFNE1 (e.g., mRNA or protein).

In an embodiment, the present invention relates to a method of evaluating whether a cancer cell is susceptible to inhibition by a cyclin-dependent kinase (CDK) inhibitor. This embodiment of the method includes measuring the amount of a biomarker transcript in the cancer cell before administering the CDK inhibitor, contacting the cancer cell with the CDK inhibitor, and measuring the amount of the biomarker transcript in the cancer cell after administering the CDK inhibitor. Comparing amount of the biomarker transcript measured after administering the CDK inhibitor to the amount of the biomarker transcript measured before administering the CDK inhibitor provides the evaluation. In an embodiment, the biomarker transcript is IFNE1 (e.g., mRNA or protein).

In a further embodiment, the present invention includes a method to identify a prognostic marker for treatment of cancer with CDK inhibitors. The method includes measuring the biomarker transcript level such as IFNE1 before and after administration of the CDK inhibitor. A biomarker transcript that shows increased expression after administration of the CDK inhibitor is identified as a prognostic marker for treatment of cancer. In yet another embodiment, the present invention includes methods to determine predisposition to resistance to treatment of cancer using CDK inhibitor. The method includes measuring the biomarker transcript such as IFNE1 before and after administration of the CDK inhibitor.

The present invention also includes methods to determine the sensitivity of cancer cells to CDK inhibitors, and methods for monitoring the pharmacodynamic action of a CDK inhibitor in surrogate tissue.

### BRIEF DESCRIPTION OF THE DRAWINGS

FIGS. 1A and 1B show the result of microarray analysis of HCT116 xenografts treated with P276-00 (P276) and Flavopiridol (FP). Supervised hierarchical clustering identifies a cluster of genes that are upregulated upon acute (A) or chronic (B), P276/Flavopiridol treatment. IFNE1 belongs to this cluster of genes and is seen to be upregulated when either of these CDK inhibitors is used to treat tumors generated by HCT116 xenografts.

FIGS. 2A and 2B show results of Real Time Quantitative Reverse Transcriptase PCR of relative IFNE1 gene expression in HCT116 xenografts treated with P276-00 and Flavopiridol. Signals were normalized to the house keeping gene, Actin (ACTB). The expression level was plotted as log fold change relative to the signal from the control group of xenografts. The values obtained from the microarray were also plotted alongside to those obtained from real time analysis. The data shows reasonable correlation between the microarray and Real Time RT-PCR analysis and therefore identifies IFNE1 as an upregulated gene upon treatment of tumors with CDK inhibitors.

FIGS. 3A, 3B and 3C show expression of IFNE1 in different cancer cell lines treated with P276-00 and Flavopiridol. The different cell lines were treated with the agent for the indicated times and the level of IFNE1 mRNA were estimated using Real Time RT-PCR. Using the Actin gene for normalization, IFNE1 expression level was plotted as log fold change relative to the signal from the control untreated cells. HCT116 (3A) HL-60 (3B) and Calu-1 (3C) cells showed a significant increase in IFNE1 level when treated with either P276-00 or FP at most time points. Of the cell lines tested, HCT116 showed a maximum increase in IFNE1 level upon P276/FP treatment.

FIG. 4 shows upregulation of IFNE1 in HCT116 cells treated with CDK inhibitors. Non-CDK inhibitors do not cause a change in IFNE1 mRNA level. HCT116 cells were treated with various CDK inhibitors [P276-00 (250 nM), Flavopiridol (200 nM), Fascaplysin (500 nM), Roscovitine (20 μM), Olomoucine (180 μM), 5,6-dichloro-1-beta-D-ribofuranosylbenzimidazole (DRB, 50 μM)] and non-CDK inhibitors [5-Fluorouracil (800 nM), Camptothecin (30 nM), Etoposide (3 μM), Vinblastin (1.5 μM), Quercitin (750 nM), Paclitaxel (600 nM), Doxorubicin (100 nM)] for 6 hours. The level of IFNE1 mRNA was estimated using Real Time RT-PCR. Using Actin gene for normalization, relative IFNE1 expression level was plotted as log fold change.

### DETAILED DESCRIPTION OF THE INVENTION

### Definitions

The term “biomarker”, as used herein, refers to a molecule or molecular species (such as a protein or gene) used to indicate or measure a biological process. Detection and analysis of a biomarker specific to a disease can aid in the identification, diagnosis, and treatment of the disease, or act as a prognostic marker for the disease. For example, the level of a particular protein found in blood may be an indicator of a specific blood-associated disorder.

For the purpose of the invention, the terms “biomarker” or “biomarker transcript” are used interchangeably.

As used herein, “an agent or therapeutic agent” used to treat cancer refers to any molecule or molecular species used to treat cancer, wherein treating cancer refers to ameliorating, mitigating or delaying the onset of the effects of cancer. The therapeutic agent may be a chemical or biochemical agent with pharmacological anti-cancer activity or chemotherapeutic activity. For example, the therapeutic agent used in the methods of the present invention can be an agent that inhibits the proliferation of cancer cells. Examples of such medicines or agents include, without limitation, inhibitors of the cyclin-dependent kinases (CDKs). The terms “anti-CDK” and “CDK inhibitor” are used interchangeably herein.

The CDK inhibitors used with the methods of the invention can be used to treat different types of cancer cells. The term “tumor” as used herein refers to a group of cells that are cancerous in origin and grow uncontrollably. Tumors from various types of cancers can be treated with CDK inhibitors.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. The terms “ribonucleic acid” and “RNA” as used herein mean a polymer composed of ribonucleotides. The terms “deoxyribonucleic acid” and “DNA” as used herein mean a polymer composed of deoxyribonucleotides. The term “oligonucleotide” as used herein means a polymer composed of either DNA or RNA, and used as probes to find a complementary sequence of DNA or RNA.

The terms “protein” or “polypeptide” are used interchangeably. They refer to a chain of two or more amino acids, which are linked together with peptide or amide bonds, regardless of post-translational modification (e.g., glycosylation or phosphorylation). Antibodies are specifically intended to be within the scope of this definition.

The phrase “substantially identical” means a sequence exhibiting at least 80%, for example, 90%, or even 95% sequence identity to the reference polypeptide sequence. The term with respect to a nucleic acid sequence shall be construed as a sequence of nucleotides exhibiting at least about 85%, for example, 90%, 95%, or even 97% sequence identity to the reference nucleic acid sequence. For polypeptides, the length of the comparison sequences will generally be at least 25 amino acids. For nucleic acids, the length will generally be at least 75 nucleotides. By “identity” is meant the percentage of nucleic acid or amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using conventional sequence analysis software.

The term “sample” as used herein relates to a material or mixture of materials, typically, although not necessarily, in fluid form, containing one or more components of interest. Samples include, but are not limited to, biological samples obtained from natural biological sources, such as cells or tissues. The samples may be derived from a tissue biopsy or another clinical procedure, and may include tumor tissue or cells extracted from mammals bearing tumor or patients having cancer. The sample may be in the form of an explant or xenograft. The tissue of the present invention may be surrogate tissue, i.e. any tissue that can be used as a substitute or replacement for tumor tissue in monitoring biological responses. The surrogate tissue may be non-proliferating peripheral mononuclear cells or proliferating cells, such as buccal mucosa tissue cells. For example, the surrogate tissue is a peripheral blood mononuclear cell(s).

As used herein, the term “mammal” refers to any animal classified as a mammal including mouse, rat and human. Preferably, the mammal is human.

As used herein, the term “patient” refers to a human being suffering from cancer and requires treatment.

For the purpose of the invention, the terms “subject”, “mammal” or “patient” are used interchangeably.

As used herein, the term “correlation” refers to the relationship between the expression or amount of one molecule and the expression or amount of another molecule. For example, the expression of a protein may be correlated to the expression of a different protein, or to the amount of agent administered to treat a particular disorder. In the method of the invention, the correlation is determined by known methods.

### METHODS OF THE INVENTION

The present invention relates to the use of a biomarker transcript as a therapeutic and prognostic marker for particular chemotherapeutic agents that target specific proteins. Specifically, the present invention involves the assessment of the expression level of a particular biomarker transcript following administration of an anti-CDK agent or CDK inhibitor to a mammal having cancer. More specifically, the present invention concerns measurement of the amount of interferon transcript (e.g., mRNA or protein). The amount of IFNE1 transcript (e.g., mRNA or protein) upon treatment with a CDK inhibitor can be compared to the amount of IFNE1 transcript before treatment. An increase in IFNE1 transcript indicates a response to CDK inhibitor. The measuring or estimating of the amount of interferon transcript can be accomplished through any of a variety of known assays.

In an embodiment, the present invention includes a method of correlating the expression of a biomarker (e.g., IFNE1 transcript) with the amount of a therapeutic agent used to treat cancer. The method includes comparing the expression of the IFNE1 transcript before the administration of the agent or therapeutic agent with the expression of that transcript after the administration of the agent or therapeutic agent. In an aspect, the agent or therapeutic agent is administered in vivo to a mammal bearing tumor, or to a patient having cancer. In another aspect, the agent or therapeutic agent is administered to tumor tissue from a mammal or a patient ex vivo, as with administration to a xenograft or explant, for example. In yet another aspect, the agent or therapeutic agent is administered to tumor tissue or cells in vitro.

In an aspect, the therapeutic agent used to treat cancer is an inhibitor of cyclin-dependent kinase, i.e. a CDK inhibitor or anti-CDK agent. CDK (Cyclin-dependent kinase) inhibitors are a new and important class of molecular candidates that target and inhibit cyclin dependent kinases (CDKs) in cells. Because CDKs play a crucial role in the control of cell cycle and because CDK activity is critical to the enhanced growth rate of cancer cells, CDK inhibitors have been developed to block the cell cycle, preferably in cancer cells. CDK inhibitors have been shown to cause apoptotic effects both as single agents and in combination with other known cytotoxic agents. In an aspect, the agent that inhibits CDK activity (CDK inhibitor or anti-CDK agent) of the present invention may be any known CDK inhibitor.

Examples of suitable CDK inhibitors include compounds such as the compounds disclosed in published PCT application WO 2004004632 and U.S. Patent Publication No. 2007015802 incorporated herein by reference. Other examples of CDK inhibitors include, without limitation, flavopiridol, roscovitine, olomoucine, 5,6-dichlorlo-1-beta-ribofuranosylbenzimidazole, fascaplysin, and synthetic compounds with anti-CDK activity, such as P276-00 ((+)-trans-2-(2-Chloro-phenyl)-5,7-dihydroxy-8-(2-hydroxymethyl-1-methyl-pyrrolidin-3-yl)-chromen-4-one hydrochloride) and other compounds described in U.S. Patent Pub. No. 20070015802, incorporated herein by reference.

The CDK inhibitors used with the methods of the invention can be used to treat a variety of different cancers, including bladder cancer, breast cancer, lung cancer, colon cancer, prostate cancer, liver cancer, pancreatic cancer, stomach cancer, testicular cancer, brain cell cancer, ovarian cancer, lymphatic cancer, skin cancer, bone cancer, and soft tissue cancer.

In an aspect, the biomarker is a cytokine such as an interferon, and in another aspect, the biomarker is a specific interferon, namely interferon epsilon or IFNE1. IFNE1 was identified as a novel interferon from sequence analysis of various interferons (Hardy et. al. Genomics 84 (2004) 331-345). The structure and mRNA expression patterns of IFNE1 suggest that it may have a function distinct from those of the other members of the human interferon family. This predicted human gene, is intron-less and is transcribed toward the telomere of HSA chromosome 9; it encodes a putative open reading frame of 208 amino acids. RT-PCR analysis showed that human IFNE1 was expressed in the human prostate cancer cell line PC-3, amnion-derived WISH cells, SK-MEL28 melanoma cells, and Daudi cells and very weakly in MCF-7 human breast cancer cells. Putative transcription factor binding sites were conserved between the human and the mouse sequence. These conserved motifs include sites for the signal transducers and activators of transcription (STATs), progesterone receptor response element (PRE), and CCAAT/enhancing protein h (CEBPh).

The inventors have identified IFNE1 as a transcript that is upregulated when human cancer cells, in culture and in xenograft mouse models, are treated with CDK inhibitors.

The length of a IFNE1 polynucleotide that can be used in the methods of the present invention is 1502 bp (accession no. NM_{—}176891), and the length of the corresponding protein sequence is 208 amino acids. The DNA sequence of this IFNE1 polynucleotide is:

| IFNE1 Nucleotide | |
|---|---|
| (SEQ ID NO: 1) | |
| 1 | cttagatatt aaactgatag gataagatat aaaataattt aagattgctg atatatgttt |
| 61 | taaaattaat tatttgctca agcatttgtg acaatttaca gttctaattg aggttttaaa |
| 121 | tttagtagtt tgtaggtatt ttaagttttg cccctgaatt ctttataggt gctgataagc |
| 181 | ctttggttaa gttttactcc atgaaagact attactgaaa aaaatgtaat ctcaataaaa |
| 241 | gaactttaat aagcttgact aaatatttag aaagcacatt gtgttcagtg aaactttgta |
| 301 | tataatgaat agaataataa aagattatgt tggatgacta gtctgtaatt gcctcaagga |
| 361 | aagcatacaa tgaataagtt attttggtac ttcctcaaaa tagccaacac aatagggaaa |
| 421 | tggagaaaat gtactctgaa caccatgaaa agggaacctg aaaatctaat gtgtaaactt |
| 481 | ggagaaatga cattagaaaa cgaaagcaac aaaagagaac actctccaaa ataatctgag |
| 541 | atgcatgaaa ggcaaacatt cactagagct ggaatttccc taagtctatg cagggataag |
| 601 | tagcatattt gaccttcacc atgattatca agcacttctt tggaactgtg ttggtgctgc |
| 661 | tggcctctac cactatcttc tctctagatt tgaaactgat tatcttccag caaagacaag |
| 721 | tgaatcaaga aagtttaaaa ctcttgaata agttgcaaac cttgtcaatt cagcagtgtc |
| 781 | taccacacag gaaaaacttt ctgcttcctc agaagtcttt gagtcctcag cagtaccaaa |
| 841 | aaggacacac tctggccatt ctccatgaga tgcttcagca gatcttcagc ctcttcaggg |
| 901 | caaatatttc tctggatggt tgggaggaaa accacacgga gaaattcctc attcaacttc |
| 961 | atcaacagct agaataccta gaagcactca tgggactgga agcagagaag ctaagtggta |
| 1021 | ctttgggtag tgataacctt agattacaag ttaaaatgta cttccgaagg atccatgatt |
| 1081 | acctggaaaa ccaggactac agcacctgtg cctgggccat tgtccaagta gaaatcagcc |
| 1141 | gatgtctgtt ctttgtgttc agtctcacag aaaaactgag caaacaagga agacccttga |
| 1201 | acgacatgaa gcaagagctt actacagagt ttagaagccc gaggtaggtg gagggactag |
| 1261 | aggacttctc cagacatgat tcttcataga gtggtaatac aatttatagt acaatcacat |
| 1321 | tgctttgatt ttgtgtatat atatatttat ctgagtttta agattgtgca tattgaccac |
| 1381 | aattgttttt attttgtaat gtggctttat atattctatc cattttaaat tgtttgtatg |
| 1441 | tcaaaataaa ttcattaata tggttgattc ttcaaaaaaa aaaaaaaaaa aaaaaaaaaa |
| 1501 | aa |

This IFNE1 polynucleotide encodes the following amino acid sequence:

IFNE1 Polypeptide
(SEQ ID NO: 2)
MIIKHFFGTVLVLLASTTIFSLDLKLIIFQQRQVNQESLKLLNKLQTLS
IQQCLPHRKNFLLPQKSLSPQQYQKGHTLAILHEMLQQIFSLFRANISL
DGWEENHTEKFLIQLHQQLEYLEALMGLEAEKLSGTLGSDNLRLQVKMY
FRRIHDYLENQDYSTCAWAIVQVEISRCLFFVFSLTEKLSKQGRPLNDM
KQELTTEFRSPR

In an embodiment, the method of the present invention includes correlating expression of a biomarker (e.g., IFNE1 transcript) with the amount of a therapeutic agent used to treat cancer. In an aspect, the method includes measuring the level of a biomarker transcript (e.g., IFNE1). The therapeutic agent is then administered, and the level of the biomarker transcript is measured again. The level of the biomarker transcript before administration of the therapeutic agent is compared to the level of the biomarker transcript after administration of the agent. The comparison is used to determine a correlation between expression of the biomarker transcript and the therapeutic agent administered. An increase in the expression of the biomarker transcript indicates that the agent or therapeutic agent is effective in treating/targeting cancer.

The measuring of level of the biomarker transcript such as IFNE1, either before or after administration of the anti-CDK agent can be conducted using known methods. In an embodiment, total RNA is extracted from mammalian tumor tissue, cells or xenografts that have been treated with the anti-CDK agent or with vehicle. The RNA is then converted to cDNA and analyzed by hybridization to a microarray to determine transcript level of IFNE1 or other biomarkers. In another embodiment, total RNA extracted from tumor tissue, cells or xenografts is used for real time quantitative polymerase chain reaction (RTQ-PCR) analysis.

The CDK inhibitors used with the methods of the invention can be administered to tumor tissue or cells in vivo, in vitro, or ex vivo, using known methods. In an embodiment, the therapeutic agent is administered by exposing tissue, cell or xenograft samples to a plurality of concentrations of the anti-CDK agent or vehicle. For example, cells maintained in culture can be treated with concentrations of flavopiridol ranging from 100 nM to 100 mM. Different concentrations can be used with different anti-CDK agents, for example, due to the differing potency or pharmacological activity (as evidenced by, for example, different IC₅₀ values) of the different agents.

In certain embodiments, tumor tissue, cells or xenografts can be exposed to anti-CDK agents for amounts of time ranging from, for example, 3 hours to 24 hours. In an embodiment, prolonged administration of CDK inhibitors can be achieved by treating tissue, cells or xenografts continuously on consecutive days. In an embodiment, the anti-CDK agents can be administered in combination with other cytotoxic agents used chemotherapeutically. These other agents include molecules that do not target CDKs, i.e. compounds that do not act as inhibitors of CDK. The amount of biomarker transcript (such as IFNE1, for example) is compared before and after administration of the agent, e.g., CDK inhibitor. In an embodiment, the amount of biomarker transcript prior to administration of the therapeutic agent to a patient can be measured by known methods. The amount of biomarker transcript in the same patient is then measured after administration of the agent. In another embodiment, measuring the amount of biomarker transcript prior to administration of the therapeutic agent to tissue, cell or xenograft samples includes exposing one group of samples to a control or vehicle, rather than to the anti-CDK agent. In another embodiment, measuring the amount of biomarker transcript prior to administration of the anti-CDK agent includes administering to the tissue, cell, or xenograft samples a different agent, i.e. a non-CDK inhibiting agent in the absence of the CDK inhibitor. In an embodiment, the correlation between expression of the biomarker transcript and administration of the anti-CDK agent provides a measure of the therapeutic response of a mammal bearing tumor, a patient having cancer, a tumor tissue, cells or xenografts, to the CDK inhibitor. The therapeutic response is a measure of the pharmacologic modulation of a target tumor or cancer. In an embodiment, the biomarker of the invention, such as IFNE1 for example, is increased subsequent to treatment with the CDK inhibitor relative to the expression of the biomarker prior to treatment. In an aspect, a two-fold increase of the biomarker indicates a positive therapeutic response to the CDK inhibitor.

In an embodiment, the present invention includes a method to determine if a cancer cell will respond to treatment with a CDK inhibitor. The method includes measuring the amount of biomarker (e.g., IFNE1) transcript in a mammal bearing tumor, a patient having cancer, or in tissue, cells or xenografts extracted from mammals bearing tumor or patients having cancer, prior to administration of the CDK inhibitor. In an aspect, the amount of biomarker transcript is measured after treatment with a vehicle, a control drug, or an agent that is not a CDK inhibitor. The CDK inhibitor is then administered in vivo, in vitro, or ex vivo, and the amount of biomarker transcript is measured again. The amount of the biomarker transcript before administration of the CDK inhibitor is compared to the level of the biomarker transcript after administration of the CDK inhibitor. In an aspect, an increase in the amount of the biomarker transcript after administration of the CDK inhibitor indicates that the cancer cell will respond to treatment with the CDK inhibitor or anti-CDK agent. In an aspect, an increase in expression of at least two-fold indicates that the cancer cell responds to treatment with CDK inhibitors. In an embodiment, the biomarker is an interferon, for example, IFNE1.

In another embodiment, the present invention includes a method to identify a prognostic marker for the treatment of cancer using a CDK inhibitor. The method includes measuring the amount of biomarker transcript in a mammal bearing tumor, a patient having cancer, or in tissue, cells or xenografts extracted from mammal bearing tumor or patients having cancer, prior to administration of the CDK inhibitor. In an aspect, the amount of biomarker transcript is measured after treatment with a vehicle, control drug, or agent that is not a CDK inhibitor. The CDK inhibitor or anti-CDK agent is then administered in vivo, in vitro or ex vivo, and the amount of biomarker transcript is measured again. The amount of biomarker transcript before administration of the therapeutic agent is compared to the amount of biomarker transcript after administration of the agent. In an aspect, a biomarker that shows increased expression after administration of the CDK inhibitor is identified as a prognostic marker for treatment of cancer with the CDK inhibitor or anti-CDK agent. In an embodiment, the biomarker is an interferon, and in another embodiment, the biomarker is IFNE1.

In yet another embodiment, the present invention includes a method to determine predisposition to resistance to treatment of cancer with CDK inhibitor. The method includes measuring the amount of biomarker transcript in a mammal bearing tumor, a patient having cancer, or in a tissue, cells or xenograft extracted from a mammal bearing tumor or patient having cancer, prior to administration of the CDK inhibitor. In an aspect, the amount of biomarker transcript is measured after treatment with a vehicle, control drug, or agent that is not a CDK inhibitor. The CDK inhibitor or therapeutic agent is then administered in vivo, in vitro or ex vivo, and the amount of biomarker transcript is measured again. The amount of the biomarker transcript before administration of the therapeutic agent is compared to the amount of the biomarker transcript after administration of the agent. In an embodiment, the biomarker is an interferon, and in another embodiment, the biomarker is IFNE1.

In an embodiment of the invention, the method includes: a) measuring IFNE1 transcript level in the tumor tissue of a mammal; b) administering to the mammal a CDK inhibitor; c) measuring the IFNE1 mRNA level in the tumor tissue wherein an increase in the level of IFNE1 mRNA in step c) compared to the level of IFNE1 mRNA in step a) indicates that the exposure of the mammal to the CDK inhibitor will produce a therapeutic response.

In a further aspect, the invention concerns a method to predict sensitivity of tumor cells to CDK inhibitors, including a) estimating IFNE1 mRNA level in the tumor tissue of a mammal; b) administering to the mammal a CDK inhibitor; c) estimating the level of IFNE1 mRNA level in the tumor tissue, wherein an increase in the level of IFNE1 mRNA in step c) compared to the level of IFNE1 mRNA in step a) indicates increased sensitivity of the tumor to a given CDK inhibitor.

In another aspect, the invention also provides a method for monitoring the pharmacodynamics of a CDK inhibitor in the surrogate tissue of a mammal including a) estimating IFNE1 mRNA level in the surrogate tissue of a mammal; b) administering to the mammal a CDK inhibitor; c) estimating the level of IFNE1 mRNA level in the surrogate tissue at one or more time points, wherein a difference in the level of IFNE1 mRNA in step c) compared to the level of IFNE1 mRNA in step a) indicates that the mammal may respond therapeutically to the method of treating cancer using CDK inhibitors.

In an embodiment, the present invention provides a method for correlating the expression of a biomarker with the administration of a chemotherapeutic agent such as a CDK inhibitor. In an aspect, the biomarker is a cytokine such as an interferon. In another aspect, the biomarker is a specific interferon, i.e. interferon epsilon (IFNE1). In yet another aspect, the biomarker is an IFNE1-like gene or polypeptide, i.e. a gene or polypeptide having biological activity similar to that of IFNE1, i.e. the polypeptide having the amino acid sequence shown in SEQ ID NO: 2. Fragments or derivatives of IFNE1 that have similar biological activity as IFNE1 are also capable of use with the methods of the invention. In another aspect, the biomarker is any polynucleotide or polypeptide that is substantially identical or homologous with the polynucleotide encoding the IFNE1 gene, as shown in SEQ ID NO: 1, or the polypeptide sequence shown in SEQ ID NO: 2, i.e. the IFNE1-like polypeptide, polynucleotide, derivative or fragment exhibits at least 70%, 80%, 90%, 95%, 97%, 98% or 99% identity or homology with the sequence shown in SEQ ID NO: 1 or 2.

In an embodiment, the present invention provides a method for monitoring the pharmacodynamics of a CDK inhibitor in the surrogate tissue of a mammal. The method includes administering the CDK inhibitor to the mammal, obtaining one or more test samples from the mammal at one or more specific time points after administering the CDK inhibitor, performing an assay to detect level of a biomarker, such as IFNE1 for example, and comparing expression level to a reference sample obtained from a mammal to which no CDK inhibitor is administered. An increase in the expression of the biomarker in the CDK inhibitor-treated samples relative to the reference sample provides a measure of the pharmacodynamic action of the CDK inhibitor.

The invention also concerns the use of IFNE1 as a biomarker for CDK inhibitors used as single agents or in combination with other cytotoxic agents. These other cytotoxic agents include non-CDK inhibitors, i.e. chemotherapeutic agents that do not target CDKs.

The mammal of the present invention may be any mammal selected from mouse, rat and human. Preferably, the mammal is human. Tissue, cells or xenografts extracted from mammals bearing tumors are capable of use with the methods of the present invention. Where the methods involve in vitro analysis of cells, a number of different cell lines can be used. Examples include, without limitation, HCT116, HL-60 and Calu-1 cell lines.

The invention is further illustrated by the following non-limiting examples.

### EXAMPLES

Compound P276-00 ((+)-trans-2-(2-Chloro-phenyl)-5,7-dihydroxy-8-(2-hydroxymethyl-1-methyl-pyrrolidin-3-yl)-chromen-4-one hydrochloride) was synthesized according to processes described in U.S. Patent Publication No. 20070015802, incorporated herein by reference. Tables 1A and 1B show the results of the microarray analysis. The analyses identify IFNE1 as a transcript that shows an increase in level of expression upon treatment with either P276-00 or FP treatment of the HCT116 xenografts in mouse models.

Table 1A shows the results of the microarray analysis. The values represents the log of median of ratio for IFNE1 gene across different xenografts treated with either P276-00 or Flavopiridol. A value above 1 indicates a two-fold increase in the gene levels. Cluster analysis identifies upregulation of IFNE1 consistently across acute treatment of HCT116 xenografts.

TABLE 1A

| | | | | | | | A23 | A5 | A2 | | | | | |
|---|---|---|---|---|---|---|---|---|---|---|---|---|---|---|
| | | | | | A20 | A11 | (P276- | (P276- | (P276- | A9 | A6 | A3 | A15 | A22 |
| UniqID | Acc | UGCluster | Name | Symbol | (P276-00) | (P276-00) | 00) | 00) | 00) | (FP) | (FP) | (FP) | (FP) | (FP) |
| 10234 | NM_176891 | Hs.682604 | Similar to | IFNE1 | 2.26 | 2.053 | 3.252 | 1.966 | 1.107 | 1.568 | 1.121 | 2.017 | 1.228 | 1.17 |
| | | | Interferon Tau | | | | | | | | | | | |

Table 1B shows the results of microarray analysis for chronic treated HCT116 xenografts. A value above 1 indicates a significant two-fold increase in the gene levels. The values are represented as described in Table 1.

TABLE 1B

| | | | | | C17 | C11 | C23 | | | |
|---|---|---|---|---|---|---|---|---|---|---|
| | | | | | (P276- | (P276- | (P276- | C15 | C9 | C6 |
| UniqID | Acc | UGCluster | Name | Symbol | 00) | 00) | 00) | (FP) | (FP) | (FP) |
| 10234 | NM_176891 | Hs.682604 | Similar to | IFNE1 | 0.7715 | 1.318 | 1.326 | 1.595 | 1.723 | 2.731 |
| | | | Interferon | | | | | | | |
| | | | Tau | | | | | | | |

### Example 1

### Cluster of Genes Up-Regulated by P276-00 and Flavopiridol Treated HCT116 Xenografts as Studied by Microarray Based Expression Profiling

### Exposure of Colon Carcinoma HCT-116 Xenografts to P276-00 and Flavopiridol

Colon carcinoma (HCT-116) cells were injected into severe combined immunodeficient (SCID) mice intraperitoneally. Xenografts were allowed to grow, typically for 7 days until the tumors reached a size of 5×5 mm before therapeutic agent administration. A total of 56 animals were used for the study. Animals that were treated with two consecutive day administrations of the agent/vehicle were considered as the acute treated group. The acute treated group of animals consisted of 8 sham treated, 10 Flavopiridol (2.5 mg/kg) treated, and P276-00 (35 mg/kg) treated mice. A similar group of animals was treated with either vehicle or agent for 6-7 days consecutively and was considered as the chronic treated group, as discussed below. The differences in the concentrations used reflect the differences in the IC₅₀ values of the two therapeutic agents.

### Preparation of RNA:

At the end of the acute/chronic treatments, the xenografts were excised from the animals and RNA was prepared using Trizol reagent (Sigma, USA) as per the manufacturer's protocol. Briefly, xenograft tissues were homogenized under liquid nitrogen and resuspended in Trizol (1 mL/mg tissue) followed by chloroform extraction. The RNA was precipitated in isopropanol and the pellet was washed using 70% ethanol. RNA samples thus obtained were resuspended in RNase-DNase free water. Quality and quantity of RNA was measured by spectrometry using ND-1000 spectrometer (Nanodrop, Wilmington, Del., USA).

### Gene Expression Profiling by Oligonucleotide Microarray:

To account for variability in gene expression in the xenografts before agent treatments, RNA samples from the respective control groups were pooled proportionally. Each of the P276/FP acute treated xenograft was, therefore, compared with pooled acute control RNA samples and the same method was adopted for the chronically treated samples. For microarray hybridization, a total of 20 μg of RNA was used for cDNA synthesis, which was indirectly labeled with Cy3 (pooled control) or Cy5 (individual treated xenograft) fluorescent dyes as described (Chinnaiyan AM et. al., Am J. Pathol. 2001 October; 159(4): 1199-209). A 23,290 oligonucleotide array (Illumina, USA) was spotted using the OmniGrid™ Genemachines. The slides were processed by standard procedures and hybridization was performed in the two-channel mode. Post hybridization, the slides were washed and scanned using GeneTAC UC-4 and images obtained were analyzed using the GenePix Pro 5.1. The median of ratios of intensities (Cy5/Cy3) obtained from the two channels was subjected to Cluster analysis and viewed using the Treeview program. Median of ratios above 2 is considered as significant upregulation of the specific gene in the drug treated xenograft and a value below 0.5 is considered as downregulation of the specific gene in the drug treated xenograft. The log values (to the base 2) for an upregulated gene would therefore be greater than 1 and that of a downregulated gene would be less than 1.

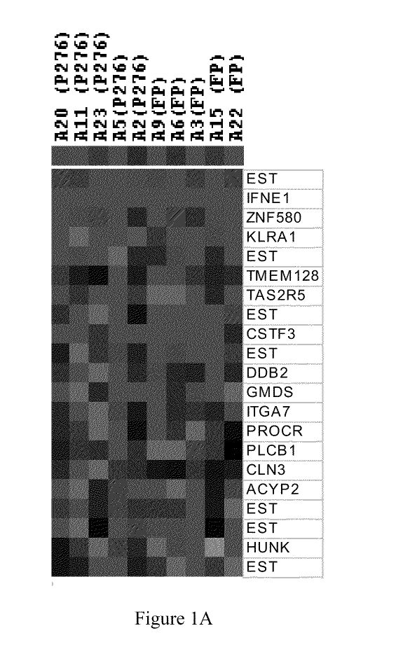

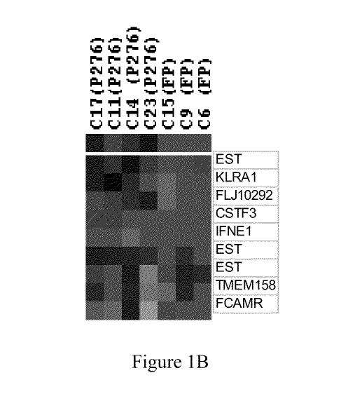

A total of 16 hybridizations representing 10 acute treatments (FIG. 1A) and 6 chronic treatments (FIG. 1B) were analyzed. Individual hybridizations were numbered according to acute (A) or chronic (C) treatment followed by the animal number and the respective agent used. For example, A20 (P276) identifies acute treated xenograft with P276-00 in the animal numbered 20.

Tables 1A and 1B show the results of the microarray analysis. The values represent the log of median of ratio for IFNE1 gene across different xenografts treated with either P276-00 or Flavopiridol. As mentioned before, a ratio value above 1 indicates a two-fold increase in the gene levels. The Cluster analysis identifies upregulation of IFNE1 consistently across both acute (Table 1A) and chronic (Table 1B) treatments of HCT116 xenografts.

### Real Time Quantitative Reverse Transcriptase PCR(RTQ-PCR):

IFNE1 expression in tumor tissues, after exposure to P276-00 or Flavopiridol, was further validated by RTQ-PCR. The primer sequences used for PCR analysis are as follows:

Forward primer sequences for IFNE1: 5′CAGCCGATGTCTGTTCTTTGTGTTC3′ (SEQ ID NO: 3)

Reverse primer sequences for IFNE1: 5′ CACCTACCTCGGGCTTCTAA3′ (SEQ ID NO: 4)

The actin (ACTB) gene is used as control for relative quantification, and the following primer sequences were used for its PCR analysis:

Forward primer sequences for ACTB: 5′GCAAAGACCTGTACGCCAACACAGT3′ (SEQ ID NO: 5)

Reverse primer sequences for ACTB: 5′AGTACTTGCGCTCAGGAGGA3′ (SEQ ID NO: 6)

An aliquot of the RNA samples used for the microarray study was also used for Quantitative Real Time Reverse TRasncriptase PCR (QRT-PCR) analysis. Briefly, cDNA was synthesized from different RNA samples using Superscript (Life Technologies, USA) and PCR was performed using SyBr green assay kit, using manufacturers protocol (Eppendorf, USA), and data analysis for QRT- PCR was carried out using the software provided by the manufacturer (Eppendorf, Westbury, N.Y., USA). All samples were assayed in duplicate.

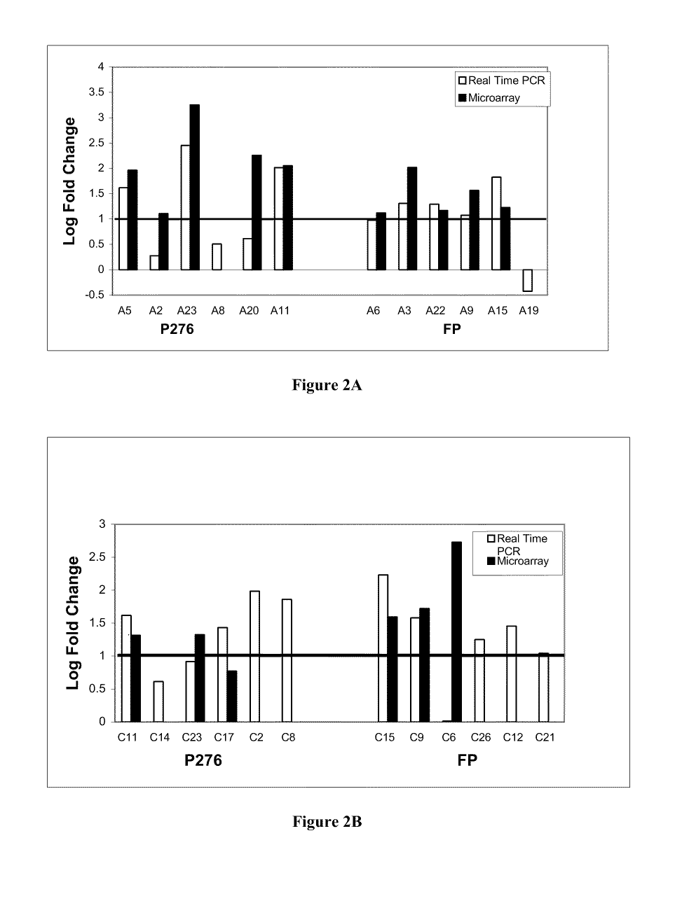

QRT-PCR shows that acute treatment with P276-00 upregulated IFNE1 level in 3 of the 6 xenografts analyzed (FIG. 2A), and in most of the xenografts tested after chronic treatment (FIG. 2B). Flavopiridol also induced IFNE1 up-regulation in most of the xenografts tested. This data, as shown in FIGS. 2A and 2B, (with IFNE1 gene expression normalized to actin levels), was in good agreement with the data obtained from the microarray experiments.

### Example 2

### IFNE1 Up-Regulation Observed After P276-00 and Flavopiridol Treatment in Different Cell Lines

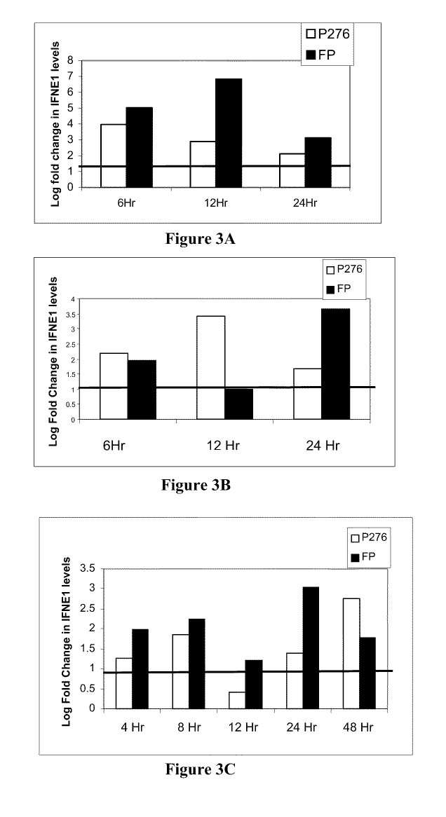

The expression of IFNE1 in response to P276-00 or Flavopiridol was also studied in different cancer cell lines such as HCT116, HL-60 (leukemia) and Calu-1. HCT116, Calu-1, H460 and HL-60 cells were obtained from ATCC and were maintained in culture medium with 10% fetal bovine serum. P276-00 and Flavopiridol were synthesized in-house and 10 mM stocks in DMSO were used for the assays. HCT116 (FIG. 3A) cells in culture were treated with P276-00 and Flavopiridol at 200 nM and 250 nM respectively for 6 hours, 12 hours and 24 hours. HL-60 (FIG. 3B) cells were treated with 300 nM of P276-00 and 100 mM of Flavopiridol for 3 hours, 6 hours, 12 hours and 24 hours and Calu-1 (FIG. 3C) cells were treated with 1.2 μM of P276-00 and 450 nM of Flavopiridol for 4 hours, 8 hours, 12 hours, 24 hours and 48 hours. RNA was prepared from the samples and QRT-PCR was performed to estimate level of IFNE1.

In HCT116, HL-60 and Calu-1 cancer cell lines, IFNE1 was up-regulated within 6 hours of treatment with either therapeutic agent (P276-00 and Flavopiridol), and there were distinct differences in the extent of up-regulation of IFNE1 between the cell lines. While the level of IFNE1 were most up-regulated in HCT116 cell line (up to 7 log fold changes by Flavopiridol in 12 hours), in HL-60 and Calu-1 cell lines IFNE1 level were increased at later time points and to a lesser extent. These results, shown in FIGS. 3A 3B and 3C, demonstrate that both P276-00 and Flavopiridol up-regulate IFNE1 transcript level across a variety of cancer cell lines but the up-regulation may be dependent on other factors.

### Example 3

### IFNE1 Upregulation as a Result of CDK Inhibition

To study if IFNE1 expression is a result of CDK inhibition, HCT116 cells were treated with a variety of CDK and non-CDK inhibitors and IFNE1 transcript level were measured using quantitative QRT-PCR. HCT116 cells were chosen for this assay because IFNE1 level seems to be most regulated in this cell line. The cells were treated with a variety of CDK inhibitors [P276-00 (250 nM), Flavopiridol (200 nM), Fascaplysin (500 nM), Roscovitine (20 μM), Olomoucine (180 μM), 5,6-dichloro-1-beta-D-ribofuranosylbenzimidazole (DRB) (50 μM)] and non-CDK inhibitors [5-Fluorouracil (800 nM), Camptothecin (30 nM), Etoposide (3 μM), Vinblastin (1.5 μM), Quercitin (750 nM), Paclitaxel (600 nM), Doxorubicin (100 nM)] for 6 hours and the IFNE1 transcript level were estimated. All inhibitors were purchased from Sigma and used at IC₅₀ values described in literature.

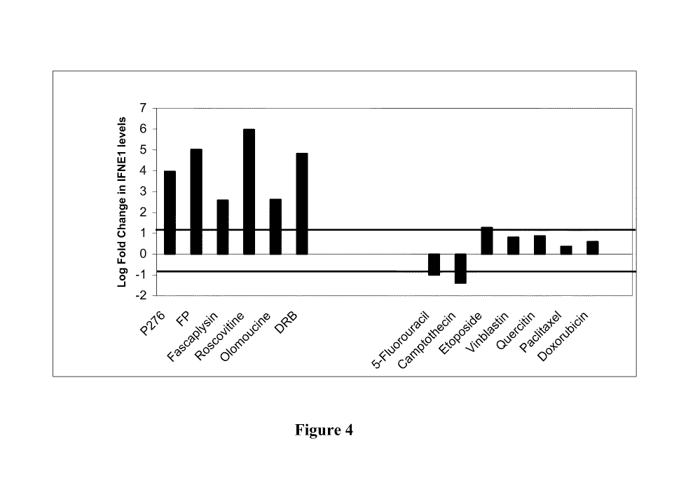

As shown in FIG. 4, inhibitors that target a variety of cyclin dependent kinases upregulate IFNE1 level, although to varied level in HCT116 cells. In contrast, non-CDK inhibitors do not upregulate IFNE1 level indicating that IFNE1 level are regulated largely by CDK inhibition. Of the CDK inhibitors used, Roscovitine potentiated IFNE1 level maximally. Most of the non-CDK inhibitors did not up-regulate IFNE1 level, indicating the role of CDK inhibition in IFNE1 expression.

The various embodiments described above are provided by way of illustration only and should not be construed to limit the claims. Those skilled in the art will readily recognize various modifications and changes that may be made to the present methods without following the example embodiments and applications illustrated and described herein, and without departing from the true spirit and scope of the following claims.

Unless defined otherwise, all technical and scientific terms used herein have the same meaning as commonly understood to one of ordinary skill in the art. Although any methods, devices and material similar or equivalent to those described herein can be used in practice or testing, the methods, devices and materials are now described.

All publications and patent applications in this specification are indicative of the level of ordinary skill in the art.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

We claim:

1. A method of evaluating whether a cyclin-dependent kinase (CDK) inhibitor will inhibit growth of a cancer cell, comprising:
measuring amount of a biomarker transcript in the cancer cell before administering the CDK inhibitor;
wherein the biomarker transcript comprises Interferon Epsilon (IFNE1) mRNA or IFNE1 protein;
contacting the cancer cell with the CDK inhibitor;
measuring amount of the biomarker transcript in the cancer cell after administering the CDK inhibitor; and
comparing the amount of the biomarker transcript measured after administering the CDK inhibitor to the amount of biomarker transcript measured before administering the CDK inhibitor;
wherein an increase in amount of the biomarker transcript indicates that the CDK inhibitor will inhibit growth of a cancer cell.

2. A method of evaluating whether a cyclin-dependent kinase (CDK) inhibitor produces a therapeutic response in treating cancer, comprising:
measuring amount of a biomarker transcript in a cancer cell from a mammal before administering the CDK inhibitor;
wherein the biomarker transcript comprises IFNE1 mRNA or IFNE1 protein;
administering the CDK inhibitor to the mammal;
measuring amount of the biomarker transcript in a cancer cell from the mammal after administering the CDK inhibitor; and
comparing the amount of the biomarker transcript measured after administering the CDK inhibitor to the amount of biomarker transcript measured before administering the CDK inhibitor;
wherein an increase in the amount of the biomarker transcript after administration of the CDK inhibitor indicates that the CDK inhibitor produces a therapeutic response in treating cancer.

3. The method of claim 1, wherein the step of contacting of the cancer cell comprises administering the CDK inhibitor to a patient having cancer.

4. The method of claim 1, wherein the step of contacting of the cancer cell comprises administering the CDK inhibitor to mammal bearing tumor.

5. The method of claim 1, wherein the step of contacting of the cancer cell comprises contacting tumor tissue with the CDK inhibitor ex vivo.

6. The method of claim 1, wherein the step of contacting of the cancer cell comprises contacting tumor tissue or cells with the CDK inhibitor in vitro.

7. The method of claim 1, wherein the cancer cell is from bladder cancer, breast cancer, lung cancer, colon cancer, prostate cancer, liver cancer, pancreatic cancer, stomach cancer, testicular cancer, brain cell cancer, ovarian cancer, lymphatic cancer, skin cancer, bone cancer, or soft tissue cancer.

8. The method of claim 1, wherein the CDK inhibitor is Flavopiridol, P276-00, Roscovitine, Olomoucine, 5,6-dichloro-1-beta-D-ribofuranosylbenzimidazole, or Fascaplysin.

9. The method of claim 1, wherein the biomarker is a polypeptide encoded by a polynucleotide having a sequence of SEQ ID NO: 1.

10. The method of claim 1, wherein the biomarker is a polypeptide encoded by a polynucleotide having a sequence:
with 80% identity to the sequence of SEQ ID NO: 1; or
with 90% identity to the sequence of SEQ ID NO: 1.

11. The method of claim 1, wherein the biomarker is a polypeptide having the sequence of SEQ ID NO: 2.

12. A method for testing whether a cyclin-dependent kinase (CDK) inhibitor produces a therapeutic response in a patient having cancer, said method comprising
a) measuring amount of IFNE1 mRNA in tumor tissue of a patient;
b) administering to the patient a cyclin-dependent kinase (CDK) inhibitor;
c) measuring IFNE1 mRNA level in the tumor tissue;
wherein an increase in the level of IFNE1 mRNA in step c) compared to the level of IFNE1 mRNA in step a) indicates that the exposure of the patient to the CDK inhibitor will produce a therapeutic response.

13. A method to predict sensitivity of a mammal bearing tumor, comprising
a) measuring amount of IFNE1 mRNA in tumor tissue of a mammal;
b) administering to the mammal a cyclin-dependent kinase (CDK) inhibitor;
c) measuring IFNE1 mRNA level in the tumor tissue;
wherein an increase in the level of IFNE1 mRNA in step c) compared to the level of IFNE1 mRNA in step a) indicates that the mammal will respond therapeutically to the method of treating cancer using the CDK inhibitor.

14. A method for identification of a prognostic marker in a surrogate tissue of a mammal, comprising:
a) measuring amount of IFNE1 mRNA in surrogate tissue of a mammal;
b) administering to the mammal a cyclin-dependent kinase (CDK) inhibitor;
c) measuring amount of IFNE1 mRNA at 3-48 hours after administration of the CDK inhibitor;
wherein an increase in IFNE1 mRNA level in step c) compared to IFNE1 mRNA level in step a) indicates that the mammal is responsive to cancer therapy involving CDK inhibitors.

15. The method of claim 12, wherein the tumor tissue is from bladder cancer, breast cancer, lung cancer, colon cancer, prostate cancer, liver cancer, pancreatic cancer, stomach cancer, testicular cancer, brain cell cancer, ovarian cancer, lymphatic cancer, skin cancer, bone cancer, or soft tissue cancer.

16. The method of claim 12, wherein the CDK inhibitor is Flavopiridol, P276-00, Roscovitine, Olomoucine, 5,6-dichloro-1-beta-D-ribofuranosylbenzimidazole, or Fascaplysin.

17. The method of claim 12, wherein the level of IFNE1 mRNA is measured in a tumor tissue extracted from the patient having cancer wherein the patient is administered with a CDK inhibitor.


SEQUENCE LISTING (6 sequences)

SEQ ID NO: 1 - DNA, 1502 bp, Homo sapiens
cttagatattaaactgataggataagatataaaataatttaagattgctgatatatgttttaaaattaattatttgctcaagcatttgtgacaatttacagttctaattgaggttttaaatttagtagtttgtaggtattttaagttttgcccctgaattctttataggtgctgataagcctttggttaagttttactccatgaaagactattactgaaaaaaatgtaatctcaataaaagaactttaataagcttgactaaatatttagaaagcacattgtgttcagtgaaactttgtatataatgaatagaataataaaagattatgttggatgactagtctgtaattgcctcaaggaaagcatacaatgaataagttattttggtacttcctcaaaatagccaacacaatagggaaatggagaaaatgtactctgaacaccatgaaaagggaacctgaaaatctaatgtgtaaacttggagaaatgacattagaaaacgaaagcaacaaaagagaacactctccaaaataatctgagatgcatgaaaggcaaacattcactagagctggaatttccctaagtctatgcagggataagtagcatatttgaccttcaccatgattatcaagcacttctttggaactgtgttggtgctgctggcctctaccactatcttctctctagatttgaaactgattatcttccagcaaagacaagtgaatcaagaaagtttaaaactcttgaataagttgcaaaccttgtcaattcagcagtgtctaccacacaggaaaaactttctgcttcctcagaagtctttgagtcctcagcagtaccaaaaaggacacactctggccattctccatgagatgcttcagcagatcttcagcctcttcagggcaaatatttctctggatggttgggaggaaaaccacacggagaaattcctcattcaacttcatcaacagctagaatacctagaagcactcatgggactggaagcagagaagctaagtggtactttgggtagtgataaccttagattacaagttaaaatgtacttccgaaggatccatgattacctggaaaaccaggactacagcacctgtgcctgggccattgtccaagtagaaatcagccgatgtctgttctttgtgttcagtctcacagaaaaactgagcaaacaaggaagacccttgaacgacatgaagcaagagcttactacagagtttagaagcccgaggtaggtggagggactagaggacttctccagacatgattcttcatagagtggtaatacaatttatagtacaatcacattgctttgattttgtgtatatatatatttatctgagttttaagattgtgcatattgaccacaattgtttttattttgtaatgtggctttatatattctatccattttaaattgtttgtatgtcaaaataaattcattaatatggttgattcttcaaaaaaaaaaaaaaaaaaaaaaaaaaaaa

SEQ ID NO: 2 - protein, 208 aa, Homo sapiens
MIIKHFFGTVLVLLASTTIFSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 3 - DNA, 25 bp, Homo sapiens
cagccgatgtctgttctttgtgttc

SEQ ID NO: 4 - DNA, 20 bp, Homo sapiens
cacctacctcgggcttctaa

SEQ ID NO: 5 - DNA, 25 bp, Homo sapiens
gcaaagacctgtacgccaacacagt

SEQ ID NO: 6 - DNA, 20 bp, Homo sapiens
agtacttgcgctcaggagga